(19) United States
(12) Patent Application Publication
(10) Pub. No.: US 2022/0108714 A1
(43) Pub. Date: Apr. 7, 2022

(54) SYSTEM AND METHOD FOR ALZHEIMER'S DISEASE DETECTION FROM SPEECH

(71) Applicant: WINTERLIGHT LABS INC., Toronto (CA)

(72) Inventors: Jekaterina NOVIKOVA, Toronto (CA); Aparna BALAGOPALAN, Toronto (CA); Benjamin EYRE, Toronto (CA); Jessica ROBIN, Toronto (CA); Frank RUDZICZ, Toronto (CA)

(21) Appl. No.: 17/320,992

(22) Filed: May 14, 2021

Related U.S. Application Data
(60) Provisional application No. 63/086,778, filed on Oct. 2, 2020.
(60) Provisional application No. 63/120,093, filed on Dec. 1, 2020.

(51) Int. Cl. G10L 25/66; G06F 16/65; G06N 20/00; G10L 15/02; G10L 15/08; G10L 15/16

(52) U.S. Cl. CPC G10L 25/66; G06F 16/65; G10L 15/16; G10L 15/02; G10L 15/08; G06N 20/00

(57) ABSTRACT

Speech impairment indicative of Alzheimer's Disease (AD) is detected from an input speech sample using a classification model comprising a fine-tuned, pre-trained Bidirectional Encoder Representations from Transformer (BERT)-based model and a classification layer, the fine-tuning using training data comprising transcribed speech, a portion of the training data identified as associated with AD and a portion of the training data identified as not associated with AD. The input need not include acoustic or temporal features. In some implementations, the classification model is augmented through the use of syntactic and word-content features extracted from the speech sample, which are concatenated with an aggregate transcript representation from the BERT-based model and provided to the classification layer.

### CROSS-REFERENCE TO RELATED APPLICATIONS

This application claims priority to U.S. Provisional Application No. 63/086,778 filed Oct. 2, 2020, and to U.S. Provisional Application No. 63/120,093 filed Dec. 1, 2020, the entireties of which are incorporated herein by reference.

### TECHNICAL FIELD

The present disclosure relates to automated detection of speech-based cognitive impairment symptoms, and in particular detection of Alzheimer's Disease (AD) from speech samples.

### TECHNICAL BACKGROUND

Research related to the automatic detection of Alzheimer's Disease (AD) is important, given the high prevalence of AD and the high cost of traditional diagnostic methods. Since AD significantly affects the content and acoustics of spontaneous speech, natural language processing (NLP) and machine learning (ML) provide promising techniques for reliably detecting AD. There has been a recent proliferation of classification models for AD, but these vary in the datasets used, model types and training and testing paradigms.

Generally, NLP and ML approaches to AD detection from speech are based on hand-crafted engineering of several hundred clinically-relevant features including certain acoustic features and linguistic features from speech transcripts. While the use engineered features makes a ML model easier to interpret, the feature engineering process is time and resource-consuming since it does require domain knowledge, may be subject to biases in data, and may result in highly relevant features being overlooked.

### BRIEF DESCRIPTION OF THE DRAWINGS

In drawings which illustrate by way of example only embodiments of the present application,

FIG. 1 is a schematic of an example data processing system implementing a system or method for AD detection employing a BERT-based classification model for detection of AD from speech samples.

FIG. 2 is a schematic of a computer-implemented system implementing a BERT-based classification model for AD detection.

### DETAILED DESCRIPTION

Alzheimer's Disease (AD) is a progressive neurodegenerative disease that causes problems with memory, thinking, and behavior. Currently, AD affects over 40 million people worldwide with high costs of acute and long-term care. Conventional forms of diagnosis are both time consuming and expensive; as a result, many people living with AD do not receive a timely diagnosis. Based on the clinical observation that information indicative of cognition can be obtained from spontaneous speech elicited using pictures, speech analysis, natural language processing (NLP), and machine learning (ML) have been to distinguish between speech from healthy and cognitively impaired participants in datasets including semi-structured speech tasks such as picture description. Such methods may serve as quick, objective, and non-invasive assessments of an individual's cognitive status which could be developed into more accessible tools to facilitate clinical screening and diagnosis.

Generally, NLP and ML approaches to AD detection from speech are based on hand-crafted engineering of several hundred clinically-relevant features including certain acoustic features (such as zero-crossing rate, Mel-frequency cepstral coefficients etc.) and linguistic features (such as proportions of various parts-of-speech (POS) tags) from speech transcripts. Engineered features have the advantage of producing more easily interpretable ML model decisions, since the selection of features is informed by domain knowledge; it was also believed to be advantageous to include representations of speech in different modalities (both acoustic and linguistic). Additionally, feature engineering may have potentially lower computational resource requirements when used with conventional ML models. However, a feature engineering process may in fact be prone to biases in data, and despite the use of domain knowledge, bears a risk of missing highly relevant features. Further, the feature engineering process can be expensive and time-consuming because it requires clinical expertise. Successful models generally involve extraction of several hundreds of features. For example, Fraser et al. (2016) extracted 370 linguistic and acoustic features from picture descriptions in the DementiaBank dataset; Balagopalan et al. (2018) augmented DementiaBank data with multi-task healthy data to improve accuracy of their ML model, employing 480 linguistic and acoustic features. DementiaBank (Becker et al., 1994) is a longitudinal dataset of speech commonly used for assessing cognitive impairment containing 473 narrative picture descriptions, where each participant describes a picture shown to them.

Transfer learning, or in other words, utilizing language representations from huge pre-trained neural models that learn robust representations for text, has become ubiquitous in NLP. A popular transfer learning model is BERT (Devlin et al., 2019), which trains “contextual embeddings” wherein a representation of a sentence (or transcript) is influenced by the context in which the words occur in sentences. This model, including its derivatives (e.g., ALBERT, RoBERTa, DistilBERT) (Lan et al., 2019; Liu, Ott et al., 2019; Sanh et al., 2019) offers enhanced parallelization and better modeling of long-range dependencies in text and as such, has achieved state-of-the-art performance on a variety of tasks in NLP. BERT uses powerful attention mechanisms to encode global dependencies between the input and output. Fine-tuning BERT for a few epochs can potentially attain good performance even on small datasets. However, unlike conventional ML models employed for AD detection, BERT takes embeddings as input rather than engineered features, and thus may be felt not to be conducive to an AD or similar impaired speech detection task. Critiques of BERT in this regard include the lack of direct interpretability and the fact that it is pre-trained on a corpus of healthy language. Further, the original version of the BERT model could take only text input, and thus could not use the acoustic modality of speech, which was generally believed important for detecting AD. Thus, BERT and similar transfer learning models may not have been previously used for developing predictive models for AD detection despite its success in NLP with healthy speech. As set out below, it was surprisingly discovered that use of a BERT model fine-tuned for the AD detection test using a relatively small AD training dataset could deliver at least as good or numerically better (more accurate) results compared to conventional ML models used for AD detection, without the need for domain knowledge or feature extraction, and using linguistic information only—without the use of acoustic features. It was also discovered that a noticeable improvement in the accuracy of a fine-tuned BERT model could be achieved with the addition of a limited set of engineered features, significantly smaller than those sets mentioned above, and again without the use of acoustic features. Eliminating feature engineering steps in whole or in part, and eliminating the extraction of acoustic features, may reduce the computational resources required to pre-process speech data as input to an AD detection ML model.

The examples below include experiments supporting the above conclusions, employing different training/test datasets. Existing studies that have addressed differences between AD and non-AD speech and worked on developing speech-based AD biomarkers are often descriptive rather than predictive. Thus, they often overlook common biases in evaluations of AD detection methods, such as repeated occurrences of speech from the same participant, variations in audio quality of speech samples, and imbalances of gender and age distribution in the used datasets. Thus, existing ML models may be prone to the biases introduced in available data. In addition, the performance of the previously developed predictive AD-detection models has been evaluated using either random train/test split or a cross-validation technique, which may result in artificially increased reported performance of ML models (i.e., overfitting) as compared to their evaluation on a held out unseen dataset, especially when it comes to smaller and unbalanced datasets. Accordingly, the systems and methods described below include models trained or fine-tuned on a new common dataset that was introduced to better compare model performance, the ADReSS Challenge (Luz et al., 2020). The ADReSS dataset comprises a demographically (age and gender) balanced speech dataset of speech from AD and non-AD participants describing a picture.

### 1. Evaluation of BERT

### Dataset

A BERT text sequence classification model was evaluated using the dataset for the AD Recognition through Spontaneous Speech (ADReSS) Challenge (Luz et al., 2020). The ADReSS dataset consists of 156 speech recordings and associated transcripts from non-AD (N=78) and AD (N=78) English-speaking participants. Speech was elicited from participants through the Cookie Theft picture from the Boston Diagnostic Aphasia exam (Goodglass et al., 2001). Speech transcripts were manually transcribed and annotated per the CHAT protocol (MacWhinney, 2000), and included speech segments from both the participant and an investigator.

In contrast to other speech datasets for AD detection such as DementiaBank's English Pitt Corpus (Becker et al., 1994), the ADReSS challenge dataset was matched for age and gender to minimize risk of bias in the prediction tasks. Characteristics of the patients in each group are set out in Tables 1-3 below. Contrasted with DementiaBank, the ADReSS dataset is significantly smaller (156 recordings/transcripts compared to 551) but demographically balanced.

TABLE 1
Basic patient characteristics in “train” and
“test” sets in ADReSS Challenge dataset.

| | | | Class | |
|---|---|---|---|---|
| Dataset | | | AD | Non-AD |
| ADReSS | Train | Male | 24 | 24 |
| | | Female | 30 | 30 |
| ADReSS | Test | Male | 11 | 11 |
| | | Female | 13 | 13 |
| DementiaBank | — | Male | 125 | 83 |
| (Becker et al., | | Female | 197 | 147 |
| 1994) | | | | |

TABLE 2
Basic patient characteristics of “train” set
in ADReSS Challenge dataset.

| | AD | | Non-AD | |
|---|---|---|---|---|
| Age | M | F | M | F |
| [50, 55) | 1 | 0 | 1 | 0 |
| [55, 60) | 5 | 4 | 5 | 4 |
| [60, 65) | 3 | 6 | 3 | 6 |
| [65, 70) | 6 | 10 | 6 | 10 |
| [70, 75) | 6 | 8 | 6 | 8 |
| [75, 80) | 3 | 2 | 3 | 2 |
| Total | 24 | 30 | 24 | 30 |

TABLE 3
Basic patient characteristics of “test” set
in ADReSS Challenge dataset.

| | AD | | Non-AD | |
|---|---|---|---|---|
| Age | M | F | M | F |
| [50, 55) | 1 | 0 | 1 | 0 |
| [55, 60) | 2 | 2 | 2 | 2 |
| [60, 65) | 1 | 3 | 1 | 3 |
| [65, 70) | 3 | 4 | 3 | 4 |
| [70, 75) | 3 | 3 | 3 | 3 |
| [75, 80) | 1 | 1 | 1 | 1 |
| Total | 11 | 13 | 11 | 13 |

Recordings were acoustically enhanced by the challenge organizers with stationary noise removal and audio volume normalization was applied across all speech segments to control for variation caused by recording conditions such as microphone placement. The speech dataset was divided into a training set and an unseen held out test set.

### Feature Extraction

The ADReSS Challenge included a baseline linguistic model. For additional comparison with BERT, classifier models were trained on manually-engineered features identified using domain knowledge. Only the portion of transcripts corresponding to the participant were used, and all participant speech segments corresponding to a single picture description were combined for extracting acoustic features.

A total of 509 engineered features were extracted from the transcripts and associated audio files. These features are summarized in Tables 4-6 below (the column # Features indicates the number of features extracted for each feature type). These features were identified as indicators of cognitive impairment in previous literature, and thus encode domain knowledge. Briefly, these features may be divided into three higher-level categories:
a. Lexico-syntactic features (297): Frequencies of various production rules from the constituency parsing tree of the transcripts (Chae and Nenkova, 2009), speech-graph based features (Mota et al., 2012), lexical norm-based features (e.g., average sentiment valence of all words in a transcript, average imageability of all words in a transcript; Warriner et al., 2013), features indicative of lexical richness, as well as syntactic features (Ai and Lu, 2010) such as the proportion of various POS-tags, and similarity between consecutive utterances.
b. Acoustic and temporal features (187): Mel-frequency cepstral coefficients (MFCCs), fundamental frequency, statistics related to zero-crossing rate, as well as proportion of various pauses (for example, filled and unfilled pauses, ratio of a number of pauses to a number of words etc.; Davis and Maclagan, 2009).
c. Semantic features based on picture description content (25): Proportions of various information content units used in the picture, identified as being relevant to memory impairment in prior literature (Croisile et al., 1996).

TABLE 4
Summary of all lexico-syntactic features extracted.

| Feature Type | # Features | Brief Description |
|---|---|---|
| Syntactic | 36 | L2 Analyzer features; utterance |
| complexity | | length, depth of syntactic parse tree |
| Production rules | 104 | Proportion of production type |
| Phrasal type ratios | 13 | Proportion, average length and |
| | | rate of phrase types |
| Lexical | 12 | Average lexical norms across |
| norm-based | | words for (e.g., imageability) |
| Lexical richness | 6 | Type-token ratios; brunet; Honor's |
| | | statistic |
| Word category | 5 | Proportion of demonstratives, |
| | | function words, light verbs and |
| | | inflected verbs, and propositions |
| Noun ratio | 3 | Ratios nouns:(nouns + verbs); |
| | | nouns:verbs; |
| | | pronouns:(nouns + pronouns) |
| Length measures | 1 | Average word length |
| Universal POS | 18 | Proportions of spaCy (spacy.io) |
| proportions | | universal POS tags |
| POS tag | 53 | Proportions of Penn Treebank |
| proportions | | POS tags |
| Local coherence | 15 | Similarity between word2vec |
| | | representations of utterances |
| Utterance | 5 | Fraction of pairs of utterances |
| distances | | below a similarity threshold |
| | | (0.5, 0.3, 0); avg/min distance |
| Speech-graph | 13 | Representing words as nodes in |
| features | | a graph and computing density, |
| | | number of loops, etc. |
| Utterance | 1 | Number of switches in verb tense |
| cohesion | | across utterances divided by total |
| | | number of utterances |
| Rate | 2 | Ratios—number of words: |
| | | duration of audio; number of |
| | | syllables:duration of speech |
| Invalid words | 1 | Proportion of words not in the |
| | | English dictionary |
| Sentiment | 9 | Average sentiment norms across |
| norm-based | | all words, noun, and verbs |

TABLE 5
Summary of all acoustic/temporal features extracted.

| Feature Type | # Features | Brief Description |
|---|---|---|
| Pauses and fillers | 9 | Total and mean duration of |
| | | pauses; long and short pause counts; |
| | | pause to word ratio; fillers (um, uh); |
| | | duration of pauses to word durations |
| Fundamental | 4 | Avg/min/max/median fundamental |
| frequency | | frequency of audio |
| Duration-related | 2 | Duration of audio and spoken |
| | | segment of audio |
| Zero-crossing rate | 4 | Avg/variance/skewness/kurtosis of |
| | | zero-crossing rate |
| MFCC | 168 | Avg/variance/skewness/kurtosis |
| | | of 42 MFCC coefficients |

TABLE 6
Summary of all semantic features extracted.

| Feature Type | # Features | Brief Description |
|---|---|---|
| Word frequency | 10 | Proportion of lemmatized words |
| | | occurrences |
| Global coherence | 15 | Cosine distances between word2vec |
| | | utterances and content units |

### Domain Knowledge-Based Classification

The lexico-syntactic, acoustic, and semantic features were extracted at transcript level and classified using four conventional machine learning models: support vector machine (SVM), neural network (NN), random forest (RF), and naïve Bayes (NB) (see scikit-learn.org/stable/). All hyperparameters were tuned to the best possible setting by searching within a grid of possible parameter values using 10-fold cross validation on the ADReSS challenge “train” set.

The SVM was trained with a radial basis function kernel with kernel coefficient (γ) 0.001, and regularization parameter set to 100.

The NN consisted of two layers of 10 units each (both the number of units and number of layers were varied while tuning for the optimal hyperparameter setting). The ReLU activation function was used at each hidden layer. The model was trained using the Adam optimization algorithm (Kingma and Ba, 2014) for 200 epochs and with a batch size of number of samples in train set in each fold. All other parameters were set to the default value.

The RF classifier fit 200 decision trees and considered \sqrt{features} when looking for the best split. The minimum number of samples required to split an internal node was 2, and the minimum number of samples required to be at a leaf node was 2. Bootstrap samples were used when building trees. All other parameters were set to the default value.

The Gaussian Naive Bayes classifier was fit with balanced priors and variance smoothing coefficient set to 1 e-10, and all other parameters set to default.

Feature selection was performed by choosing the top-k number of features, based on ANOVA F-value between label/features. The number of features was jointly optimized with the classification model parameters.

### Transfer Learning-Based Classification

A combined text sequence classification model (i.e., base BERT model with classification layer) was implemented using the open-source PyTorch library (pytorch.org). In order to leverage the language information encoded by BERT (Devlin et al., 2019), the pre-trained BERT model weights were used to initialize the classification model. All experiments are based on the bert-base-uncased variant (Devlin et al., 2019), which consists of 12 layers, each having a hidden size of 768 and 12 attention heads, with a maximum input length of 512 tokens. Linear scheduling was used for the learning rate, which was initially set to 2e-5, and the Adam optimization algorithm was used. Fine-tuning for AD detection employed cross-entropy loss.

While the base BERT model was pre-trained with (healthy) sentence pairs, the input used to fine-tune the model for performing AD detection consisted of speech transcripts from the ADReSS train set. Each speech transcript comprised several transcribed utterances, which were tokenized and delimited with start and separator special tokens from the BERT vocabulary at the beginning and end of each utterance, respectively (i.e., [CLS] and [SEP]), following Liu and Lapata (2019). This ensured that utterance boundaries were encoded, since cross-utterance information such as coherence and utterance transitions is considered important for reliable AD detection.

Following Devlin et al. (2019), an aggregate transcript representation was extracted from the base BERT model for each transcript. This aggregate representation was the final hidden state corresponding to the first start ([CLS]) token in the transcript, which is an embedding pooling information across all tokenized units in the transcript. This final hidden state summarized information across all tokens in the transcript using BERT's self-attention mechanism. This embedding was passed to the classification layer (Devlin et al., 2019; Wolf et al., 2019).

In tuning the hyperparameters, the number of epochs was optimized at 10 by varying the number from 1 to 12 during cross validation. As noted above, Adam optimization and linear scheduling for the learning rate were used. The learning rate and other parameters were set based on prior work on fine-tuning BERT (Devlin et al., 2019; Wolf et al., 2019).

### Evaluation

Two cross-validation (CV) strategies were used: leave-one-subject-out CV (LOSO CV) and 10-fold CV at transcript level. Evaluation metrics with LOSO CV were determined for all models except fine-tuned BERT for direct comparison to ADReSS Challenge baselines. No LOSO CV was performed for fine-tuned BERT due to computational constraints; instead, 10-fold CV to compare the feature-based classification models with fine-tuned BERT. Values of performance metrics for each model were averaged across three runs with different random seeds in all cases.

Three predictions were generated with different seeds from each hyperparameter-optimized classifier trained on the complete ADReSS train set, then a majority prediction was produced to avoid overfitting. Task performance was evaluated primarily using accuracy scores, since the ADReSS train and test sets were known to be demographically balanced. Table 7 sets out the classification performance of all models evaluated on the ADReSS train set via 10-fold CV. Precision, recall, specificity, and F1 are also included with respect to the positive class (AD).

TABLE 7
10-fold CV results averaged across three runs with
different random seeds on the ADReSS train set.

| Model | # Features | Accuracy | Precision | Recall | Specificity | F1 |
|---|---|---|---|---|---|---|
| SVM | 10 | 0.796 | 0.81 | 0.78 | 0.82 | 0.79 |
| NN | 10 | 0.762 | 0.77 | 0.75 | 0.77 | 0.76 |
| RF | 50 | 0.738 | 0.73 | 0.76 | 0.72 | 0.74 |
| NB | 80 | 0.750 | 0.76 | 0.74 | 0.76 | 0.75 |
| BERT | — | 0.818 | 0.84 | 0.79 | 0.85 | 0.81 |

As can be seen from Table 7, BERT numerically outperformed all domain knowledge-based machine learning models with respect to all metrics, with an average accuracy of 81.8%. SVM was found to be the best-performing domain knowledge-based model. However, accuracy of the fine-tuned BERT model was not significantly higher than that of the SVM classifier based on an Kruskal-Wallis H-test (H=0.4838, p>0.05). The Kruskal-Wallis H-test was employed here and with the performance-comparisons discussed below, since it was observed that accuracy was not normally distributed on varying the random seed during training or inference.

The performance results on the unseen, held out ADReSS challenge test set are set out in Table 8 below:

TABLE 8
AD detection results on unseen, held out ADReSS test set averaged over three
runs with different random seeds.

| Model | # Features | Accuracy | Precision | Recall | Specificity | F1 | AUROC |
|---|---|---|---|---|---|---|---|
| Baseline | — | 0.755 | — | — | — | 0.7800 | — |
| SVM | 10 | 0.8125 | 0.8000 | 0.8333 | 0.7917 | 0.8124 | 0.8125 |
| NN | 10 | 0.7708 | 0.7671 | 0.7778 | 0.7639 | 0.7708 | 0.7708 |
| RF | 50 | 0.7569 | 0.8033 | 0.6806 | 0.8333 | 0.7555 | 0.7500 |
| NB | 80 | 0.7292 | 0.7895 | 0.6250 | 0.8333 | 0.7262 | 0.7292 |
| BERT | — | 0.8332 | 0.8389 | 0.8333 | 0.8333 | 0.8327 | 0.8333 |

As can be seen in Table 8, the results follow the trend of the cross-validated performance in Table 7 in terms of accuracy, with the fine-tuned BERT model outperforming the best feature-based classification model SVM with an accuracy of 83.33%, but not significantly so (H=2.4, p>0.05). The accuracy with the BERT-based classification model described above ranged between 85.14 and 81.25%.

These results demonstrate that a fine-tuned BERT model may perform the same, or numerically better, than classifier models conventionally selected for AD and similar detection tasks, which are reliant on engineered features defined by domain knowledge. Further, the feature-based and BERT-based classification models described above performed significantly better than the linguistic baseline provided in the ADReSS challenge, showing the importance of linguistic features for detecting AD-related differences.

### Feature Differentiation Analysis

A feature differentiation analysis was performed to identify the most differentiating features between AD and non-AD speech in the ADReSS training set. In order to study statistically significant differences in linguistic/acoustic phenomena, independent t-tests were performed between feature means for each class in the ADReSS training set, following the methodology followed by Balagopalan et al. (2020). Eighty-seven features were found to be significantly different between the two groups at p<0.05. Seventy-nine of these were text-based lexico-syntactic and semantic features, while eight were acoustic (including temporal). These eight acoustic features included the number of long pauses, pause duration, and mean/skewness/variance-statistics of various MFCC coefficients. However, after Bonferroni correction for multiple testing, it was found that 13 features were significantly different between AD and non-AD speech at p<9e-5, and none of these features were acoustic (including temporal features). In Table 9 below, μ_{AD} and μ_{non-AD} show the means of the 13 significantly different features at p<9e-5 after Bonferroni correction for the AD and non-AD group, respectively.

TABLE 9
Feature differentiation analysis results for the most important
features in the ADReSS train set.

| Feature | Feature Type | μ_{AD} | μ_{non-AD} |
|---|---|---|---|
| Average cosine distance between | Semantic | 0.91 | 0.94 |
| utterances | | | |
| Fraction of pairs of utterances below a | Semantic | 0.03 | 0.01 |
| similarity threshold (0.5) | | | |
| Cosine distance between word2vec | Semantic | 0.46 | 0.38 |
| utterances and content units | | | |
| Distinct content units mentioned: total | Semantic | 0.27 | 0.45 |
| content units | | | |
| Distinct action content units mentioned: | Semantic | 0.15 | 0.30 |
| total content units | | | |
| Distinct object content units mentioned: | Semantic | 0.28 | 0.47 |
| total content units | | | |
| Average word length (in letters) | Lexico-syntactic | 3.57 | 3.78 |
| Proportion of pronouns | Lexico-syntactic | 0.09 | 0.06 |
| Ratio (pronouns):(pronouns + nouns) | Lexico-syntactic | 0.35 | 0.23 |
| Proportion of personal pronouns | Lexico-syntactic | 0.09 | 0.06 |
| Proportion of adverbs | Lexico-syntactic | 0.06 | 0.04 |
| Proportion of adverbial phrases | Lexico-syntactic | 0.02 | 0.01 |
| amongst all rules | | | |
| Proportion of non-dictionary words | Lexico-syntactic | 0.11 | 0.08 |

The features that differentiate the AD and non-AD groups largely indicate semantic impairments in AD, reflected in the types of words used and the content of their picture descriptions. Many of the differentiating features reflect findings in prior literature suggesting that despite the ADReSS dataset being more demographically balanced, many of the previous findings are maintained. In addition, the differentiating features are consistent with other previous clinical literature documenting decreased specificity and information content in AD. For example, the features relating to the content units in the picture and the cosine similarity between utterances and picture content units show that the picture descriptions produced in AD have fewer relevant content words and that the words used are less semantically related to the themes of the picture. Lower average cosine distance in AD signifies more repetition in speech. These findings are also consistent with previous studies reporting reduced information content and coherence in AD. Other differentiating features related to the use of shorter words, and increased use of pronouns, adverbs, and words not found in the dictionary. These features may all reflect the use of less specific and simpler language, and reflecting previous findings of decreased specificity of language in AD. However, while Fraser et al. (2016) found differences in acoustic features, none of those findings survived Bonferroni correction can be seen above. Without wishing to be bound by theory, these findings may indicate that a demographically balanced dataset reduces the acoustic differences between groups. Further, without wishing to be bound by theory, the finding that linguistic (semantic and lexico-syntactic) features are particularly differentiating between AD and non-AD classes may explain why the BERT-based classification model, trained and fine-tuned only on linguistic features, attained performance well above random chance.

### Interpretation of Attention Patterns in BERT-Based Models

Additionally, multi-scale attention visualizations of the BERT classification model fine-tuned for AD detection as described above were produced using the BertViz library (Vig, 2019), since attention patterns may assist in interpreting model decisions, given that self-attention is an important component of BERT-based models. Attention weights for the first [CLS] token were visualized for both AD and healthy speech transcripts (the visualization may be found in Balagopalan et al., 2021, the entirety of which is incorporated herein by reference). It was found that attention weights were often attributed to a few important information content units such as such as “water,” “boy,” etc., which have been identified to be important speech indicators of AD in prior work (Fraser et al., 2016). Sometimes, attention weights were attributed to pauses and fillers, such as “uh” and “um”, and to the sentence separator tokens. Without wishing to be bound by theory, this may represent counting the number of utterances in the transcript.

### 2. Augmentation of Fine-Tuned BERT-Based Models

The possibility of augmenting a BERT-based model was studied by executing probing tasks on embedded representations extracted from different BERT layers.

### Dataset

The same initial BERT-based classification model was also fine-tuned for the task of AD detection at utterance level using the DementiaBank dataset. For fine-tuning the BERT model, each transcript was divided into individual utterances. Each utterance was treated as a sample, similar to the methodology followed by Karlekar et al. (2018). However, only data from the picture description task was used for the intended classification task, because other speech tasks were exclusively performed by participants with AD. Each sentence spoken by the participant was associated with the diagnosis label of the participant, thus increasing the sample size to 5103 utterances, which were split at about 82%/9%/9% for train/validation/test respectively. The utterances were bounded by a start [CLS] and end [SEP] token, as before. Table 10 below sets out the split details.

TABLE 10
Train/val/test splits of DementiaBank.

| Data Subset | # Utterances |
|---|---|
| Train | 4269 |
| Validation | 429 |
| Test | 409 |

### Probing Intermediate Representations of Fined-Tuned BERT Model

The representations of the first classification ([CLS]) token from each layer of the fine-tuned BERT classification model were probed (Jawahar et al., 2019). Multi-Layer Perceptrons (MLPs) were trained using these embedded representations as input to predict the following five properties:
a. WordContent: Given a (word, sentence) pair, predict if the word is present in the sentence or not.
b. SentenceLength: Given a sentence, predict its length in number of word tokens.
c. TopConstituents: Given a sentence, predict the sequence of top-level constituents in its syntax tree.
d. TreeDepth: Given a sentence, predict the length of its syntactic parse-tree.
e. BiGramShift: Given a sentence, predict whether adjust words are inverted or word order is preserved (For example, inversion is seen “This an is example sentence.”).

These properties were selected since it had been found that features similar to these properties were important for AD detection from picture descriptions (Fraser et al., 2016; Yancheva et al., 2015). For example, variations in proportions of various production rules from the constituency parse representation, which are features of syntactic type, were found to be an important characteristic of impaired speech, and presence of informative content words or units such as “cookie” or “boy” while describing the picture has been mentioned as an important characteristic. Such informative content words are identified by clinicians, and are not arbitrarily defined.

Gridsearch hyperparameter optimization was performed to arrive at the optimal parameter settings using the validation set. It was observed that performance on the syntactic task of tree-depth prediction was low, as was the performance on the word content prediction task. These probing results are set out in Table 11 below, with boldface indicating the worst performance in each feature type.

TABLE 11
Probing results on BERT fine-tuned on AD classification task.

| Linguistic Feature | Highest Accuracy | Layer | Feature Type |
|---|---|---|---|
| WordContent | 22.47 | 4 | Surface |
| SentenceLength | 92.81 | 3 | Surface |
| TopConstituents | 80.86 | 7 | Syntactic |
| TreeDepth | 36.14 | 6 | Syntactic |
| BiGramShift | 85.42 | 12 | Syntactic |

### Feature Extraction and Classification Results

Based on the above results, it was identified that engineered features capturing presence of informative content words in utterances, and syntactic tree depths may improve AD detection performance in combination with BERT. Thus, 119 features were extracted at utterance level: 117 syntactic and two word-content based features.

The two word-content features extracted were:
a. A Boolean indicating the presence of informative content units relating to the DementiaBank “Cookie Theft” picture: “boy”, “son”, “brother”, “girl”, “daughter”, “sister”, “female”, “woman”, “adult”, “grownup”, “mother”, “lady”, “cookie”, “biscuit”, “treat”, “cupboard”, “closet”, “shelf”, “curtain”, “drape”, “drapery”, “dish”, “cup”, “counter”, “apron”, “dishcloth”, “dishrag”, “towel”, “rag”, “cloth”, “jar”, “container”, “plate”, “sink”, “basin”, “washbasin”, “washbowl”, “washstand”, “tap”, “faucet”, “stool”, “seat”, “chair”, “water”, “dishwater”, “liquid”, “window”, “frame”, “glass”, “floor”, “outside”, “yard”, “outdoors”, “backyard”, “garden”, “driveway”, “path”, “tree”, “bush”, “exterior”, “kitchen”, “room”, “take”, “steal”, “fall”, “ignore”, “notice”, “daydream”, “pay”, “overflow”, “spill”, “wash”, “dry”, “sit”, “stand”.
b. Total number of information content units in each utterance.

The 117 syntactic features included depth-related features of the constituency parse representations, the height of the constituency parse-tree, proportion of verb-phrases, and proportion of production rules of type “adjective phrase followed by adjective”.

Several input settings were compared to see the effect of these features:
a. NN with FS1: A Neural Network (NN) classifier using the set of all 119 features.
b. Fine-tuned BERT: Fine-tuning a BERT sequence classification model, where a linear layer maps the concatenation of the final hidden layer representation from BERT to binary class labels (Wolf et al., 2019).
c. BERT+FS1: Fine-tuning a BERT sequence classification model in which a linear layer maps the concatenation of the final hidden layer representation from BERT and the feature vector to binary class labels.

It was found that fine-tuned BERT models were able to perform well above chance for the utterance-level AD detection task. It was also observed that the augmented BERT model—a combination of the BERT classification model with the selected engineered features—attained the highest accuracy, about 13% accuracy points higher than using classification models using features alone, and about 5% higher than fine-tuned BERT alone. These results are set out in Table 12 below, where FS1 denotes the feature set discussed above, and boldface indicates the highest performance.

TABLE 12
Results on the AD detection task.

| Model | Accuracy | Sensitivity | Specificity |
|---|---|---|---|
| NN + FS1 | 0.63 | 0.64 | 0.62 |
| Finetuned BERT | 0.71 | 0.62 | 0.79 |
| BERT + FS1 | 0.76 | 0.63 | 0.86 |

While the engineered features were selected using probing tasks, the identification of additional features may be replaced by other augmentation methods, such as generating explanations (Mothilal et al., 2020).

### Example Implementation

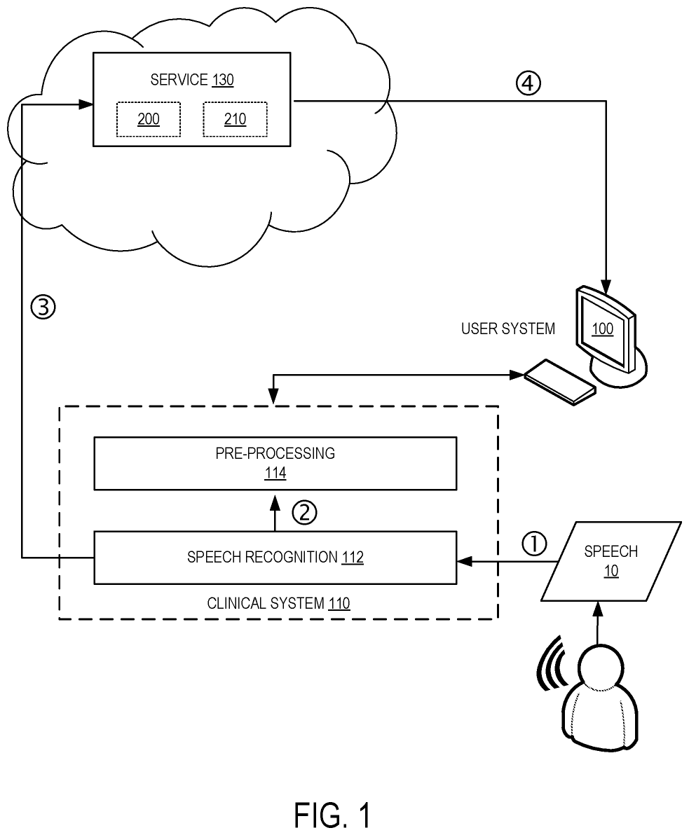

FIG. 1 illustrates an example data processing environment or system in which a BERT-based classification model may be implemented, whether for training or deployment. In this example, detection of impaired speech may be carried out using a cloud-based or otherwise remote analysis service 130 communicating with one or more client systems 150 over a network. Such a remote service 130 is preferably operated in compliance with any applicable privacy legislation, such as the United States Health Insurance Portability and Accountability Act of 1996 (HIPAA).

Individual user systems 100 may be computer systems in clinical or consultation settings, communicating with the remote service 130 over a wide area network (e.g., the Internet). It is contemplated that users may implement clinic or patient management software locally, and that preferably, personally identifying information (PII) of a patient—which can include recorded utterances—is stored securely, and even locally, to avoid accidental dissemination of PII by third-party services. This is reflected in the example data processing system of FIG. 1, in which a patient's speech is received and recorded 100 using any appropriate recording technology, and provided to a clinical system 110. The clinical system 110 may comprise the clinic or patient management software, or a dedicated application that communicates with the remote analysis service 130. The clinical system 110 may comprise a further remote server system or cloud-based system, accessible by the user system 100 via a network. The clinical system 110 may be operated or hosted by a third party provider, but in some implementations, it may be hosted locally in the clinical setting, e.g., co-located with the user system 100.

The clinical system 110 in this example is configured to receive the recorded speech (1), convert the recorded speech to text using a suitable speech recognition module 112 as would be known to those skilled in the art, and to provide the recognized speech (2) to a pre-processing module 114. The pre-processing functions executed by the module 114 may comprise speech recognition and/or feature extraction to identify linguistic or acoustic features, if feature extraction is required to generate a feature set as input to a ML classification system. The pre-processing functions may alternatively or additionally comprise speech recognition and/or tokenization or other preparation of the recognized speech data. The audio data corresponding to the recorded speech may also be pre-processed, for example to remove noise. Alternatively, in some implementations, a transcript of the subject's speech may be produced manually, and the featurization may be carried out manually as well. This may occur locally (e.g., in the clinical setting). The results of the pre-processing are transmitted (3) to the remote analysis service 130. Alternatively, no pre-processing is carried out, and instead the recorded speech 10 is transmitted (3) to the remote analysis service 130. Any data transmitted to the remote service 130 may be completely or partially anonymized, for example identified only using a patient identification number.

The remote analysis service 130 may implement both training (which, in the context of a BERT-based classification model, may comprise primarily or only fine-tuning and hyperparameter optimization since the initial parameters of a pre-trained BERT model may be employed) and classification functions employing module 200, which executes the model. It will be appreciated, however, that that actual training and/or fine-tuning of the classification model may be carried out outside the illustrated data processing system, with the resultant model packaged and imported into the remote analysis service 130 for execution by a classification module 210, e.g., a server providing a REST (REpresentational State Transfer) web service). In a classification task, the remote service 130 receives the speech input 10 or the pre-processed speech input data; performs any pre-processing that may still be required; then applies the resultant data as input to the module 200 or 210 to produce a resultant classification output, which may then be transmitted (4) over the network to the user system 100.

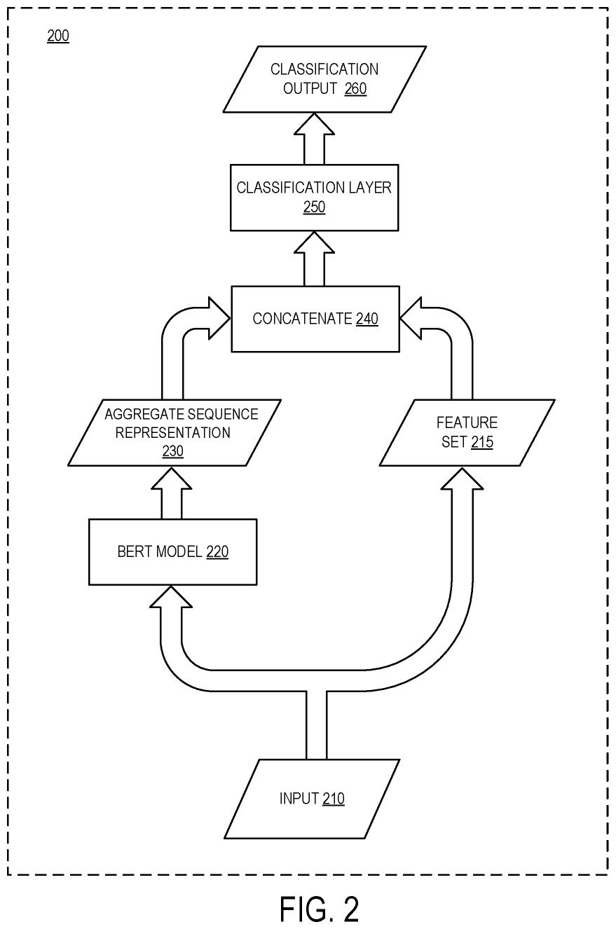

FIG. 2 provides a possible high-level schematic of the module 200 executing the BERT based classification model described above for the purpose of training/fine-tuning and/or AD classification. In a first implementation, input 210 (e.g., tokenized utterances as described above) are provided to the BERT model 220, which may be the same bert-base-uncased variant or another variant; those skilled in the art will also understand that the BERT model need not be based on a publicly available pre-trained model, but may also be pre-trained by the operator of the remote system 130, likely on a large healthy language corpus, for example as described in Devlin et al. (2019). As with the examples described above, this may be implemented using open source libraries known to those skilled in the art. Still further, other BERT-based models or variants may be employed for the AD detection task, such as, but not limited to, BERT, ALBERT, DistilBERT, and RoBERTa and their variants, which are encoder-only Transformer-based models with similar architecture and similarly pre-trained (e.g., for Masked Language and Next-Sentence-Prediction), with or without augmentation with the feature vector described above. References to a BERT-based classification model or architecture herein encompass all such models or variants suitable for text sequence classification unless otherwise indicated. The classification layer may be a linear or a non-linear layer, such as a fully connected layer.

In the AD task without augmentation, an aggregate sequence representation 230 for each input transcript is obtained, and would be provided as input directly to the classification layer 250 (not indicated in FIG. 2) to provide a classification output 260. If the augmentation described above is implemented, a feature set 215 is also extracted from the input 210, for example as described above; the resultant vector is then concatenated 240 and the result provided as input to the classification layer 250 to obtain the output 260.

Thus, there is provided a computer-implemented method, system and computer-readable medium configured for detecting speech impairment indicative of Alzheimer's Disease (AD) from a speech sample, by providing input transcribed speech to a classification model, the input comprising at least one utterance, the classification model comprising a fine-tuned, pre-trained Bidirectional Encoder Representations from Transformer (BERT)-based model and a classification layer, the fine-tuning using training data comprising transcribed speech, a portion of the training data identified as associated with AD and a portion of the training data identified as not associated with AD; and obtaining a classification of the transcribed speech input as either associated with AD or not associated with AD.

In one aspect, the BERT-based model is pre-trained on healthy speech sentence pairs. The BERT-based model may be an original BERT model or a variant.

In another aspect, input does not comprise acoustic or temporal features. Such features may comprise information about pauses and fillers, fundamental frequency, duration of audio, duration of spoken segment of audio, zero-crossing rate statistics, and mel-frequency cepstral coefficient statistics.

In a further aspect, the input comprises a plurality of utterances.

The training data may be demographically balanced, and may be significantly smaller than a training data set used to pre-train the BERT-based model. The training data may comprise a set of transcribed speeches each comprising at least one utterance bounded by a start token and an end token. The training data may comprise sets comprising a plurality of utterances.

The utterances of the input and training data may be delimited or bounded by a start token, which may be a [CLS] token, and an end token, which may be a [SEP] token. Each utterance is tokenized.

In an aspect, speech not associated with AD is healthy speech.

In another aspect, obtaining the classification comprises: obtaining an aggregate transcript representation of the input from the BERT-based model; and providing the aggregate transcript representation to the classification layer to obtain the classification.

In a further aspect, each utterance is bounded by a start token and an end token as described above, and the aggregate transcript representation is a final hidden state corresponding to the first start token of the input.

The classification layer may be a linear layer or a non-linear layer. It may be a dense layer, being fully-connected with its respective preceding (input) and following (output) layers.

In still another aspect, a feature vector is obtained for the input comprising syntactic features and word content features, wherein obtaining the classification comprises providing a concatenation of an aggregate transcript representation from the BERT-based model and the feature vector to the classification layer to obtain the classification.

The feature vector may comprise utterance-level syntactic features and utterance-level word-content features. The syntactic features may comprise information about syntactic tree depths associated with the input. These features may be depth-related features of constituency parse representations, height of a constituency parse-tree, proportion of verb-phrases, and proportion of production rules of type “adjective phrase followed by adjective”. The word-content features may comprise a value or flag, such as a Boolean, indicating presence of informative content units and a total number of informative content units in each utterance. The informative content units may be clinically defined.

As set out above, the present disclosure also provides use of a computer-implemented classification module comprising a fine-tuned BERT-based model and a classification layer for detecting speech impairment indicative of AD from input transcribed speech.

In one aspect, the classification module is configured to obtain a feature vector for the input comprising syntactic features and word content features which is concatenated with an aggregate transcript representation from the BERT-based model and provided to the classification layer to obtain a classification of the input as indicative of AD or not indicative of AD. The BERT-based model, input, utterances, classification layer and feature vector may be as described above. The pre-training and fine-tuning of the BERT-based model may be as described above.

The examples and embodiments are presented only by way of example and are not meant to limit the scope of the subject matter described herein. Individual features of each example or embodiment presented above may be combined, in whole or in part, with individual features of other examples or embodiments. Some steps or acts in a process or method may be reordered or omitted, and features and aspects described in respect of one embodiment may be incorporated into other described embodiments. Variations of these examples will be apparent to those in the art and are considered to be within the scope of the subject matter described herein.

The data employed by the systems, devices, and methods described herein may be stored in one or more data stores. The data stores can be of many different types of storage devices and programming constructs, including but not limited to RAM, ROM, flash memory, programming data structures, programming variables, and so forth. Code adapted to provide the systems and methods described above may be provided on many different types of computer-readable media including but not limited to computer storage mechanisms (e.g., CD-ROM, diskette, RAM, flash memory, computer hard drive, etc.) that contain instructions for use in execution by one or more processors to perform the operations described herein. The media on which the code may be provided is generally considered to be non-transitory or physical.

Computer components, software modules, engines, functions, and data structures may be connected directly or indirectly to each other in order to allow the flow of data needed for their operations. The data processing and computer systems described above may be provided in a single location, or may be distributed in a cloud environment, using techniques known to those skilled in the art. Those skilled in the art will know how to implement the particular examples described above in the systems and methods of FIGS. 1 and 2. Further, the systems and methods described above may be implemented in a standalone or dedicated application or environment or detecting AD from speech, or alternatively integrated into a more complex system that carries out additional functions, such as speech recognition for other functions or purposes, and/or a clinical or patient management system. or Various functional units have been expressly or implicitly described as modules, engines, or similar terminology, in order to more particularly emphasize their independent implementation and operation. Such units may be implemented in a unit of code, a subroutine unit, object, applet, script or other form of code. Such units may also be implemented in hardware circuits comprising custom circuits or gate arrays; field-programmable gate arrays; programmable array logic; programmable logic devices; commercially available logic chips, transistors, and other such components. As will be appreciated by those skilled in the art, where appropriate, functional units need not be physically located together, but may reside in different locations, such as over several electronic devices or memory devices, capable of being logically joined for execution. Functional units may also be implemented as combinations of software and hardware, such as a processor operating on a set of operational data or instructions.

Use of any particular term should not be construed as limiting the scope or requiring experimentation to implement the claimed subject matter or embodiments described herein. Any suggestion of substitutability of the data processing or computer systems or environments for other implementation means should not be construed as an admission that the invention(s) described herein are abstract, or that the data processing systems or their components are non-essential to the invention(s) described herein.

A portion of the disclosure of this patent document contains material which is or may be subject to one or more of copyright, design, or trade dress protection, whether registered or unregistered. The rightsholder has no objection to the reproduction of any such material as portrayed herein through facsimile reproduction of this disclosure as it appears in the Patent Office records, but otherwise reserves all rights whatsoever.

### REFERENCES

Haiyang Ai and Xiaofei Lu. A web-based system for automatic measurement of lexical complexity. In 27th Annual Symposium of the Computer-Assisted Language Consortium (CALICO-10). Amherst, Mass. June 1010 (pp. 8-12).
Aparna Balagopalan, Jekaterina Novikova, Frank Rudzicz, and Marzyeh Ghassemi. The effect of heterogeneous data for Alzheimer's disease detection from speech. NeurlPS Workshop on Machine Learning for Health ML4H, 2018. URL arxiv.org/abs/1811.12254.
Aparna Balagopalan, Benjamin Eyre, Frank Rudzicz, and Jekaterina Novikova. To BERT or Not To BERT: Comparing Speech and Language-based Approaches for Alzheimer's Disease Detection. Proceedings of 21st Annual Conference of the International Speech Communication Association, INTERSPEECH, 2020. URL arxiv.org/abs/2008.01551.
Aparna Balagopalan, Benjamin Eyre, Jessica Robin, Frank Rudzicz, and Jekaterina Novikova. Comparing Pre-trained and Feature-Based Models for Prediction of Alzheimer's Disease Based on Speech. Frontiers in Aging Neuroscience, 2021. doi: 10.3389/fnagi.2021.635945.
James T Becker, Francois Boiler, Oscar L Lopez, Judith Saxton, and Karen L McGonigle. The natural history of Alzheimer's Disease: Description of study cohort and accuracy of diagnosis. Archives of Neurology, 51(6):585-594, 1994.
Jieun Chae and Ani Nenkova. Predicting the fluency of text with shallow structural features: Case studies of machine translation and human-written text. 2009. repository.upenn.edu/cgi/viewcontent.cgi?article=1763&context=cis_papers
Bernard Croisile, Bernadette Ska, Marie-Josee Brabant, Annick Duchene, Yves Lepage, Gilbert Aimard, and Marc Trillet. Comparative study of oral and written picture description in patients with Alzheimer's disease. Brain and language 53, no. 1 (1996): 1-19.
Boyd H. Davis and Margaret Maclagan. Examining pauses in Alzheimer's discourse. American Journal of Alzheimer's Disease & Other Dementias® 24, no. 2 (2009): 141-154.
Jacob Devlin, Ming-Wei Chang, Kenton Lee, and Kristina Toutanova. BERT: Pretraining of deep bidirectional transformers for language understanding. In Proceedings of the 2019 Conference of the North American Chapter of the Association for Computational Linguistics: Human Language Technologies, Volume 1 (Long and Short Papers), pages 4171-4186, 2019.
Kathleen C Fraser, Jed A Meltzer, and Frank Rudzicz. Linguistic features identify Alzheimer's Disease in narrative speech. Journal of Alzheimer's Disease, 49(2):407-422, 2016.
Ganesh Jawahar, Benoît Sagot, and Djamé Seddah. What does BERT learn about the structure of language? In Proceedings of the 57th Annual Meeting of the Association for Computational Linguistics, pages 3651-3657, 2019.
Sweta Karlekar, Tong Niu, and Mohit Bansal. Detecting linguistic characteristics of Alzheimer's dementia by interpreting neural models. arXiv preprint arXiv:1804.06440, 2018.
Diederik P Kingma and Jimmy Ba. Adam: A method for stochastic optimization. arXiv preprint. arXiv:1412.6980 (2014).
Zhenzhong Lan, Mingda Chen, Sebastian Goodman, Kevin Gimpel, Piyush Sharma, and Radu Soricut. Albert: A lite bert for self-supervised learning of language representations. arXiv preprint. arXiv:1909.11942 (2019).
Yang Liu and Mirella Lapata. Text Summarization with Pretrained Encoders. In Proceedings of the 2019 Conference on Empirical Methods in Natural Language Processing and the 9th International Joint Conference on Natural Language Processing (EMNLP-IJCNLP), pp. 3721-3731. 2019.
Yinhan Liu, Myle Ott, Naman Goyal, Jingfei Du, Mandar Joshi, Danqi Chen, Omer Levy, Mike Lewis, Luke Zettlemoyer, and Veselin Stoyanov. Roberta: A robustly optimized bert pretraining approach.” arXiv preprint. arXiv:1907.11692 (2019).
Saturnino Luz, Fasih Haider, Sofia de la Fuente, Davida Fromm, and Brian MacWhinney. Alzheimer's Dementia Recognition through Spontaneous Speech: The ADReSS Challenge. INTERSPEECH 2020. homepages.ed.ac.uk/sluzfil/ADReSS/.
Natalia B Mota, Nivaldo A P Vasconcelos, Nathalia Lemos, Ana C. Pieretti, Osame Kinouchi, Guillermo A. Cecchi, Mauro Copelli, and Sidarta Ribeiro. Speech graphs provide a quantitative measure of thought disorder in psychosis. PloS one 7, no. 4 (2012): e34928.
Ramaravind K Mothilal, Amit Sharma, and Chenhao Tan. Explaining machine learning classifiers through diverse counterfactual explanations. In Proceedings of the 2020 Conference on Fairness, Accountability, and Transparency, pages 607-617, 2020.
Victor Sanh, Lysandre Debut, Julien Chaumond, and Thomas Wolf. “DistilBERT, a distilled version of BERT: smaller, faster, cheaper and lighter.” arXiv preprint arXiv:1910.01108 (2019).
Jesse Vig. (2019). A multiscale visualization of attention in the transformer model. arXiv preprint. arXiv:1906.05714.
Amy Beth Warriner, Victor Kuperman, and Marc Brysbaert. Norms of valence, arousal, and dominance for 13,915 English lemmas. Behavior research methods 45, no. 4 (2013): 1191-1207.
Thomas Wolf, Lysandre Debut, Victor Sanh, Julien Chaumond, Clement Delangue, Anthony Moi, Pierric Cistac, Tim Rault, Remi Louf, Morgan Funtowicz, et al. HuggingFace's transformers: State-of-the-art natural language processing. ArXiv, pages arXiv-1910, 2019.
Maria Yancheva, Kathleen C Fraser, and Frank Rudzicz. Using linguistic features longitudinally to predict clinical scores for Alzheimer's Disease and related dementias. In Proceedings of SLPAT 2015: 6th Workshop on Speech and Language Processing for Assistive Technologies, pages 134-139, 2015.

1. A method of detecting speech impairment indicative of Alzheimer's Disease (AD) from a speech sample, the method comprising:
providing input transcribed speech to a classification model, the input comprising at least one utterance, the classification model comprising a fine-tuned, pre-trained Bidirectional Encoder Representations from Transformer (BERT)-based model and a classification layer, the fine-tuning using training data comprising transcribed speech, a portion of the training data identified as associated with AD and a portion of the training data identified as not associated with AD; and
obtaining a classification of the transcribed speech input as either associated with AD or not associated with AD.

2. The method of claim 1, wherein the BERT-based model is pre-trained on healthy speech sentence pairs.

3. The method of claim 1, wherein the input does not comprise acoustic or temporal features.

4. The method of claim 3, wherein the acoustic or temporal features comprise information about pauses and fillers, fundamental frequency, duration of audio, duration of spoken segment of audio, zero-crossing rate statistics, and mel-frequency cepstral coefficient statistics.

5. The method of claim 1, wherein the input comprises a plurality of utterances.

6. The method of claim 1, wherein each utterance is bounded by a start token and an end token.

7. The method of claim 1, wherein the classification layer comprises either a linear layer or a non-linear layer.

8. The method of claim 1, further comprising obtaining a feature vector for the input comprising syntactic features and word content features, and wherein obtaining the classification comprises providing a concatenation of an aggregate transcript representation from the BERT-based model and the feature vector to the classification layer to obtain the classification.

9. The method of 8, wherein the feature vector comprises utterance-level syntactic features and utterance-level word-content features.

10. The method of claim 9, wherein the syntactic features comprise depth-related features of constituency parse representations, height of a constituency parse-tree, proportion of verb-phrases, and proportion of production rules of type “adjective phrase followed by adjective”.

11. The method of claim 9, wherein the word-content features comprise a Boolean indicating presence of informative content units and a total number of informative content units in each utterance.

12. A computer system comprising at least one processor and memory configured to implement detecting speech impairment indicative of Alzheimer's Disease (AD) from a speech sample, comprising:
providing input transcribed speech to a classification model, the input comprising at least one utterance, the classification model comprising a fine-tuned, pre-trained Bidirectional Encoder Representations from Transformer (BERT)-based model and a classification layer, the fine-tuning using training data comprising transcribed speech, a portion of the training data identified as associated with AD and a portion of the training data identified as not associated with AD; and
obtaining a classification of the transcribed speech input as either associated with AD or not associated with AD.

13. The computer system of claim 1, wherein the input does not comprise acoustic or temporal features.

14. The computer system of claim 13, wherein the acoustic or temporal features comprise information about pauses and fillers, fundamental frequency, duration of audio, duration of spoken segment of audio, zero-crossing rate statistics, and mel-frequency cepstral coefficient statistics.

15. The computer system of claim 12, wherein the input comprises a plurality of utterances.

16. The computer system of claim 12, wherein each utterance is bounded by a start token and an end token.

17. The computer system of claim 12, the detecting further comprising obtaining a feature vector for the input comprising syntactic features and word content features, and wherein obtaining the classification comprises providing a concatenation of an aggregate transcript representation from the BERT-based model and the feature vector to the classification layer to obtain the classification.

18. The computer system of 17, wherein the feature vector comprises utterance-level syntactic features and utterance-level word-content features.

19. A non-transitory computer-readable medium storing code which, when executed by at least one processor of a computer system, causes the system to implement detecting speech impairment indicative of Alzheimer's Disease (AD) from a speech sample, comprising:
providing input transcribed speech to a classification model, the input comprising at least one utterance, the classification model comprising a fine-tuned, pre-trained Bidirectional Encoder Representations from Transformer (BERT)-based model and a classification layer, the fine-tuning using training data comprising transcribed speech, a portion of the training data identified as associated with AD and a portion of the training data identified as not associated with AD; and
obtaining a classification of the transcribed speech input as either associated with AD or not associated with AD.

20. The non-transitory computer-readable medium of claim 19, the detecting further comprising obtaining a feature vector for the input comprising syntactic features and word content features, and wherein obtaining the classification comprises providing a concatenation of an aggregate transcript representation from the BERT-based model and the feature vector to the classification layer to obtain the classification.
